(19) United States
(12) Patent Application Publication
(10) Pub. No.: US 2023/0242854 A1
(43) Pub. Date: Aug. 3, 2023

(54) TUBULAR PACKED-BED CELL CULTURE VESSELS, SYSTEMS, AND RELATED METHODS

(71) Applicant: CORNING INCORPORATED, CORNING, NY (US)

(72) Inventor: Todd Marshall Wetherill, Painted Post, NY (US)

(21) Appl. No.: 18/011,793

(22) PCT Filed: Jun. 24, 2021
(86) PCT No.: PCT/US2021/038801

§ 371 Date: Dec. 20, 2022

Related U.S. Application Data
(60) Provisional application No. 63/046,080, filed on Jun. 30, 2020.

(51) Int. Cl. C12M 1/12

(52) U.S. Cl. CPC C12M 23/06; C12M 25/02; C12M 25/14; C12M 25/06

(57) ABSTRACT

A cell culture system is provided that includes a bioreactor vessel having an interior void defining a cell culture space, an inlet fluidly connected to a first end of the cell culture space, and an outlet fluidly connected to a second end of the cell culture space; and at least one cell growth element disposed in the cell culture space. The cell growth element includes a cell culture substrate surrounding a support element extending in a direction from the first end to the second end of the cell culture space.

This is a national stage application under 35 U.S.C. § 371 of International Application No. PCT/US2021/038801, filed Jun. 24, 2021, which claims the benefit of priority under 35 U.S.C. § 119 of U.S. Provisional Application Ser. No. 63/046,080 filed on Jun. 30, 2020, the content of which is relied upon and incorporated herein by reference in its entirety.

### FIELD OF THE DISCLOSURE

This disclosure general relates to vessels and systems for culturing cells, as well as methods for culturing cells. In particular, the present disclosure relates to cell culturing vessels and substrate incorporated therein, and methods of culturing cells using such vessels and substrates.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and Corning HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing

Another example of a high-density culture system for anchorage dependent cells is a packed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or substrate systems to entrap the cells have been previously disclosed U.S. Pat. Nos. 4,833,083; 5,501,971; and 5,510,262. Packed bed matrices usually are made using porous particles as substrates or non-woven microfibers of polymer. Such bioreactors function as recirculation flow-through bioreactors. One of the significant issues with such bioreactors is the non-uniformity of cell distribution inside the packed bed. Essentially, these packed beds function as depth filters with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of existing packed bed systems is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the packed bed during cells harvesting step. It is based on loosening the packed bed substrate and agitation or stirring of packed bed particles to allow porous matrices to collide and thus detach the cells. However, this approach is laborious and may cause significant cells damage, thus reducing overall cell viability.

An example of a packed-bed bioreactor currently on the market is the iCellis® produced by Pall Corporation. The iCellis uses small strips of cell substrate material consisting of randomly oriented fibers in a non-woven arrangement. These strips are packed into a vessel to create a packed bed. However, as with similar solutions on the market, there are drawbacks to this type of packed-bed substrate. Specifically, non-uniform packing of the substrate strips creates visible channels within the packed bed, leading to preferential and non-uniform media flow and nutrient distribution through the packed bed. Studies of the iCellis® have noted a “systemic inhomogeneous distribution of cells, with their number increasing from top to bottom of fixed bed,” as well as a “nutrient gradient . . . leading to restricted cell growth and production,” all of which lead to the “unequal distribution of cells [that] may impair transfection efficiency.” (Rational plasmid design and bioprocess optimization to enhance recombinant adeno-associated virus (AAV) productivity in mammalian cells. Biotechnol. J. 2016, 11, 290-297). Studies have noted that agitation of the packed bed may improve dispersion, but would have other drawbacks (i.e., “necessary agitation for better dispersion during inoculation and transfection would induce increased shear stress, in turn leading to reduced cell viability.” Id.). Another study noted of the iCellis® that the uneven distribution of cells makes monitoring of the cell population using biomass sensors difficult (“ . . . if the cells are unevenly distributed, the biomass signal from the cells on the top carriers may not show the general view of the entire bioreactor.” Process Development of Adenoviral Vector Production in Fixed Bed Bioreactor: From Bench to Commercial Scale. Human Gene Therapy, Vol. 26, No. 8, 2015).

In addition, because of the random arrangement of fibers in the substrate strips and the variation in packing of strips between one packed bed and another of the iCellis®, it can be difficult for customers to predict cell culture performance, since the substrate varies between cultures. Furthermore, the packed substrate of the iCellis® makes efficiently harvesting cells very difficult or impossible, as it is believed that cells are entrapped by the packed bed.

Roller bottles have several advantages such as ease of handling, and ability to monitor cells on the attachment surface. However, from a production standpoint, the main disadvantage is the low surface area to volume ratio while the roller bottle configuration occupies a large area of manufacturing floor space. Various approaches have been used to increase the surface area available for adherent cells in a roller bottle format. Some solutions have been implemented in commercially available products, but there remains room for improvement to increase roller bottle productivity even further. Traditionally, a roller bottle is produced as a single structure by a blow-molding process. Such manufacturing simplicity enables economic viability of roller bottles in bioprocessing industry. Some roller bottle modifications to increase the available surface area for cell culturing can be achieved without changing manufacturing process, however only marginal increase of modified roller bottle surface area is obtained. Other modifications of the roller bottle design add significant complexity to manufacturing processes making it economically unviable in the bioprocessing industry. It is desirable therefore to provide roller bottle with increased surface area and bioprocessing productivity, while using the same blow-molding process for its manufacturing.

Being able to scale from a small-scale bioreactor to a larger scale, such as for pilot line developing or production level, has also proved difficult or inefficient with existing technologies. Thus, it would be desirable to provide a system or platform that enables culturing cells at various scales with predictable and consistent results.

While manufacturing of viral vectors for early-phase clinical trials is possible with existing platforms, there is a need for a platform that can produce high-quality product in greater numbers in order to reach late-stage commercial manufacturing scale.

There is a need for cell culture matrices, systems, and methods that enable culturing of cells in a high-density format, with uniform cell distribution, and easily attainable and increased harvesting yields.

### SUMMARY

According to an embodiment of this disclosure, a cell culture system is provided. The system includes a bioreactor vessel having an interior void defining a cell culture space, an inlet fluidly connected to a first end of the cell culture space, and an outlet fluidly connected to a second end of the cell culture space. The system further includes at least one cell growth element disposed in the cell culture space. The cell growth element includes a cell culture substrate surrounding a support element extending in a direction from the first end to the second end of the cell culture space.

According to various aspects of the above embodiment, the cell culture system includes a sheet of cell culture substrate material that is wrapped or wound around the support element. The cell culture substrate can include a woven substrate material having a plurality of interwoven fibers with surfaces configured for adhering cells thereto. The system can further include a plurality of cell growth elements disposed in the cell culture space and aligned in the direction from the first end to the second end of the cell culture space. The plurality of cell growth elements can be removably attached to the cell culture space such that the cell culture system can accommodate various numbers of cell growth elements during cell culture.

According to some aspects of embodiments, the central support is tubular with a peripheral wall surrounding a hollow core. The peripheral wall includes a plurality of perforations fluidly connecting an interior of the central support to an exterior of the central support. The hollow core of the central support is fluidly connected to the inlet, and the cell culture system includes a fluid flow path that comprises flowing from the inlet, then through the hollow core, then radially out from the central support through the plurality of perforations, then through the cell culture substrate, and then out through the outlet.

The system can further include an inlet plenum fluidly connected to and disposed between the inlet and the cell culture space. In some embodiments, the system further includes a perforated inlet plate disposed between the inlet plenum and the cell culture space. The perforated inlet plate includes a plurality of perforations fluidly connecting the inlet plenum directly to the hollow core at a first end of the central support. The system can further include an outlet plenum fluidly connected to and disposed between the cell culture space and the outlet. A perforated outlet plate may be disposed between the cell culture space and the outlet plenum, where the perforated outlet plate includes a plurality of perforations fluidly connecting a portion of the cell culture space having the exterior of the central support to the outlet plenum. As an aspect of embodiments, the central support is attached to a second end of the central support. The hollow core is not open at the second end of the central support such that the hollow core is not directly fluidly connected to the outlet plenum via the second end of the central support.

As a further aspect of the above embodiments, the system further includes an inlet manifold disposed in the inlet plenum. The inlet manifold is fluidly connected to the inlet and configured to distribute fluid evenly throughout the inlet plenum or evenly to the perforated inlet plate. An outlet manifold can be disposed in the outlet plenum. The outlet plenum is fluidly connected to the outlet and configured to direct fluid exiting the cell culture space to the outlet.

The at least one cell culture element can have a cylindrical shape. In embodiments, the at least one cell culture element has an attachment means for attaching the cell culture substrate to the central support. In various embodiments, the cell culture space has a volume of at least about 50 mL, at least about 100 mL, at least about 200 mL, at least about 300 mL, at least about 500 mL, at least about 1 L, at least about 2 L, at least about 3 L, at least about 10 L, at least about 20 L, at least about 30 L, at least about 40 L, at least about 50 L, from about 50 mL to about 500 mL, from about 1 L to about 10 L, or from about 10 L to about 50 L. The cell culture system can include from about 7 cell culture elements to about 130 cell culture elements. In some embodiments, the cell culture substrate includes a stack or roll of cell culture substrate material without any other solid material between adjacent layers of the cell culture substrate.

According to another embodiment of this disclosure, a cell culture vessel is provided. The vessel includes a bioreactor vessel having an interior void defining a cell culture space, an inlet fluidly connected to a first end of the cell culture space, and an outlet fluidly connected to a second end of the cell culture space. The vessel further includes an inlet plenum fluidly connected to and disposed between the inlet and the cell culture space; an outlet plenum fluidly connected to and disposed between the cell culture space and the outlet; and a perforated inlet plate disposed between the inlet plenum and the cell culture space, the perforated inlet plate having at least one perforation. The cell culture space is arranged to house at least one cell growth element therein, the at least one cell growth element having a porous cell culture substrate surrounding a perforated central tube, and the at least one perforation of the perforated inlet plate fluidly connects the inlet plenum directly to a hollow center of the perforated central tube when the at least one cell growth element is disposed in the cell culture space.

According to aspects of some embodiments, the vessel further includes a perforated outlet plate disposed between the cell culture space and the outlet plenum, the perforated outlet plate having at least one perforation. The at least one perforation of the perforated outlet plate fluidly connects a portion of the cell culture space comprising an exterior of the perforated central tube when the at least one cell growth element is disposed in the cell culture space. The perforated outlet plate can include at least one attachment site for attaching the at least on cell culture element. The vessel can further include an inlet manifold disposed in the inlet plenum, the inlet manifold fluidly connected to the inlet and configured to distribute fluid evenly throughout the inlet plenum or evenly to the perforated inlet plate. According to some embodiments, the vessel further includes an outlet manifold disposed in the outlet plenum, the outlet plenum fluidly connected to the outlet and configured to direct fluid exiting the cell culture space to the outlet. The cell culture vessel is configured to operate in culturing cells while housing any of a variety of numbers of cell culture elements. The cell culture space can have a volume of at least about 50 mL, at least about 100 mL, at least about 200 mL, at least about 300 mL, at least about 500 mL, at least about 1 L, at least about 2 L, at least about 3 L, at least about 10 L, at least about 20 L, at least about 30 L, at least about 40 L, at least about 50 L, from about 50 mL to about 500 mL, from about 1 L to about 10 L, or from about 10 L to about 50 L. In some embodiments, the cell culture space is arranged to house from about 7 cell culture elements to about 130 cell culture elements.

### BRIEF DESCRIPTION OF THE DRAWINGS

FIG. 1 is an isometric view of a cell culture system having a cell culture vessel with multiple cell culture elements, according to one or more embodiments of this disclosure.

FIG. 2 is a cross-section view of the cell culture system of FIG. 1, according to one or more embodiments of this disclosure.

FIG. 3 is an example of a central support member for the cell culture element, according to one or more embodiments of this disclosure.

FIG. 4 is a partially see-through isometric view of a cell culture element, including a central support and cell culture substrate material, according to one or more embodiments of this disclosure.

FIG. 5 is a detail view of an inlet plenum and manifold of a bioreactor system, according to one or more embodiments of this disclosure.

FIG. 6 is a detail view of an outlet plenum and manifold of a bioreactor system, according to one or more embodiments of this disclosure.

FIG. 7 is a detail view of flow paths into and through the cell culture space, according to one or more embodiments of this disclosure.

FIG. 8 is a detail view of flow paths through and out of the cell culture space, according to one or more embodiments of this disclosure.

FIG. 9 is a simulation of flow vectors from an inlet plenum into the cell culture elements, according to one or more embodiments of this disclosure.

FIG. 10 is another detail view of flow paths into and through the cell culture space, according to one or more embodiments of this disclosure.

FIG. 11 is another detail view of flow paths through and out of the cell culture space, according to some embodiments.

FIG. 12 is a plan view of a perforated outlet plate according to embodiments.

FIG. 13 is an isometric view of a cell culture system according to another embodiment of this disclosure.

FIG. 14 is a cross-section view showing the flow-path through the system, according to one or more embodiments of this disclosure.

FIG. 15 shows multiple vessels in a stacked arrangement, according to one or more embodiments of this disclosure.

FIG. 16 shows bioreactor vessels of different sizes as a example of the scalability of one or more embodiments of this disclosure.

FIG. 17 is a view of the smallest vessel from FIG. 16.

FIG. 18 is a view of the vessel from FIG. 16 with the outer cover removed to show the cell culture elements housed by the cell culture space, according to one or more embodiments.

FIG. 19 is a cross-section view of the vessel from FIG. 19.

FIG. 20 shows the modeled flow characteristics of the vessel from FIGS. 17-19, with a volume of 200 mL.

FIG. 21 shows the modeled flow characteristics near the inlet of the vessel from FIGS. 17-19.

FIG. 22 shows an isometric view of a roll of cell culture substrate material, for example, according to one or more embodiments.

FIG. 23 shows a cell culture substrate in a rolled cylindrical configuration, according to one or more embodiments.

FIG. 24 shows a perspective view of a three-dimensional woven cell culture substrate, according to one or more embodiments of this disclosure.

FIG. 25 is a plan view of the substrate of FIG. 24.

FIG. 26 is a cross-section along line A-A of the substrate in FIG. 25.

FIG. 27A shows an example of a cell culture substrate of a first size, according to some embodiments.

FIG. 27B shows an example of a cell culture substrate of a second size, according to some embodiments.

FIG. 27C shows an example of a cell culture substrate of a third size, according to some embodiments.

FIG. 28A shows a perspective view of a multilayer cell culture substrate, according to one or more embodiments.

FIG. 28B shows a plan view of a multilayer cell culture substrate, according to one or more embodiments.

FIG. 29 shows a cross-section view along line B-B of the multilayer cell culture substrate of FIG. 28B, according to one or more embodiments.

FIG. 30 is a schematic representation of a cell culture system, according to one or more embodiments.

FIG. 31 is a detailed schematic of a cell culture system, according to one or more embodiments.

FIG. 32 shows a process flow chart for culturing cells on a cell culture system, according to one or more embodiments.

FIG. 33 shows an operation for controlling a perfusion flow rate of a cell culture system, according to one or more embodiments.

FIG. 34 is a schematic of a seed train process, according to one or more embodiments of this disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure relate to cell culture systems, including bioreactor vessels and cell culture substrates, and methods of culturing cells using such a substrate and bioreactor systems.

In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the substrate. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. This non-uniform distribution of the cells inside of the packed-bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Medium flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide cell growth substrates, matrices of such substrates, and/or packed-bed cell culture vessels and systems using such substrates that enable efficient and high-yield cell culturing for anchorage-dependent cells and production of cell products (e.g., proteins, antibodies, viral particles). The cell culture systems described herein use discrete cell culture element having a cell growth substrate and a flow path, channel, or tube to help distribute cell culture media throughout the volume of cell culture substrate. The discrete nature of the cell culture elements provides a quantifiable and uniform unit of cell culture substrate that can be used alone or in multiples to achieve the desired yield of cell culture or cell culture products, allowing for a scalable and predictable system. The design of the cell culture systems disclosed herein allow for the individual cell culture elements to be adequately and uniformly seeded, cultured, and/or harvested.

Embodiments of the cell culture substrate include a porous cell-culture substrate made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the cell culture system. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture substrate having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In one embodiment, a substrate is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture substrate disclosed herein supports attachment and proliferation of anchorage dependent cells in a high volumetric density format. Uniform cell seeding of such a substrate is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed substrate, and can avoid formation of large and/or uncontrollable 3D cellular aggregates with limited nutrient diffusion and increased metabolite concentrations. Thus, the substrate eliminates diffusional limitations during operation of the bioreactor. In addition, the substrate enables easy and efficient cell harvest from the bioreactor. The structurally defined substrate of one or more embodiments enables complete cell recovery and consistent cell harvesting from the packed bed of the bioreactor. The cell culture substrate according to one or more embodiments of this disclosure is more fully described in related U.S. patent application Ser. No. 16/781,685, which is incorporated herein by reference in its entirety.

According to some embodiments, a method of cell culturing is also provided using bioreactors with the cell culture substrate for bioprocessing production of therapeutic proteins, antibodies, viral vaccines, or viral vectors.

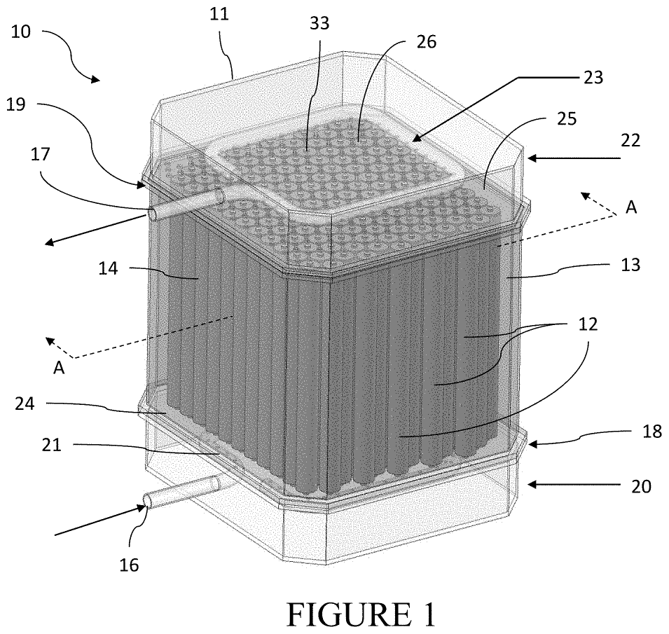

As shown in FIG. 1, a cell culture system 10 according to one or more embodiments of this disclosure includes bioreactor vessel 11 and at least one cell growth element 12. The bioreactor vessel 11 has an interior void defining a cell culture space 13 for housing the at least one cell growth element 12. The cell culture space 13 may house multiple cell culture elements 12 during cell culture. Thus, the cell culture space 13 is sized to accommodate an appropriate number of cell culture elements 12 for the desired application. However, an aspect of one or more embodiments allows for the number of cell culture elements 12 to be added to or removed from the bioreactor vessel 11. This flexibility in the number of cell culture elements 12 allows for scaling the yield of a given bioreactor vessel 11. In addition, one or more cell culture elements 12 can be removed, in whole or part, during cell culture to allow for sampling. Sampling is an important feature for users who wish to know how their culture is progressing. The cell culture element 12 itself includes a cell culture substrate 14 surrounding a support element 15, which are described in more detail below. Each cell culture element 12 is arranged to extend from a first end 18 of the cell culture space 13 to a second end 19 of the cell culture space 13, and substantially parallel to an overall flow direction F of media through the cell culture space 13.

The cell culture space 13 is fluidly connected to an inlet 1164 and an outlet 17. The inlet 16 is configured to provide at least one of cells, cell culture media, and cell nutrients to the cell culture space, and at least one of the cells, cell culture media, and cell nutrients can exit the cell culture system 10 via the outlet 17. In addition, cell byproducts or harvested cells can be withdrawn through either the inlet 16 or the outlet 17, depending on the system design. As shown in FIG. 1, according to some embodiments, the bioreactor vessel 11 can include an inlet plenum 20 disposed between the inlet 16 and the cell culture space 13. The inlet plenum 20 can help to even distribute media across the width of the bioreactor vessel 11 before the media enters the cell culture space 13. In this way, media can be evenly distributed to each cell culture element 12. To aid in even distribution of media and/or cells, an inlet manifold 21 can be provided in the inlet plenum 20. The inlet manifold 21 is fluidly connected to the inlet 16 and includes a number of spaced openings or connectors for distributing fluid from the inlet 16 to one or more areas of the inlet plenum 20 or the cell culture space 13. Similarly, the bioreactor vessel 11 can further include an outlet plenum 22 disposed between the cell culture space 13 and the outlet 17. The outlet plenum 22 optionally includes an outlet manifold 23 for directing fluid, cells, or byproducts to the outlet 17.

As will be discussed in further detail below, the bioreactor vessel 11 can further include at least one of a perforated inlet plate 24 and a perforated outlet plate 25, which separate the cell culture space 13 from the inlet plenum 20 and the outlet plenum 22, respectively. The perforations in the perforated inlet and outlet plates 24, 25 can be configured for both fluid flow into and out of the cell culture space 13, respectively, and for attachment or alignment of the cell culture elements 12. For example, in FIG. 1, a top flange 16 of each cell culture element 12 can be seen resting on top of the perforated outlet plate 25.

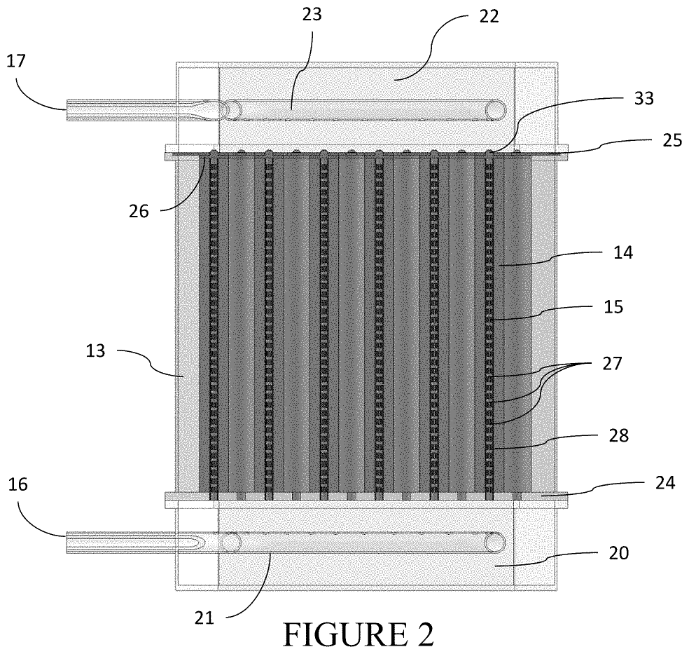

FIG. 2 shows a cross-section view of the bioreactor vessel 11 of FIG. 1 taken along line A-A. The portions of the bioreactor vessel 11 described above can be clearly seen in the cross-section of FIG. 2. In addition, the cross-section reveals a cross-section view of some of the cell culture elements 12, revealing the support element 15 within the cell culture substrate 14. Each support element 15 has a number of perforations 27 in an outer wall 28 of the support element 15. These perforations 27 allow for media to flow from the hollow interior space 29 of the support element 15 to an exterior space 30 of the support element 15. As shown, the support elements 26 extend through the perforated outlet plate 25 to where the top flange 26 of the support element 26 rests on top of the perforated outlet plate 25. Optionally, the support elements 26 may also extend into or through the perforations in the perforated inlet plate 24 to keep the cell culture elements properly aligned and to allow media to flow from the inlet plenum 20 to the interior space 29 of the cell culture elements 12.

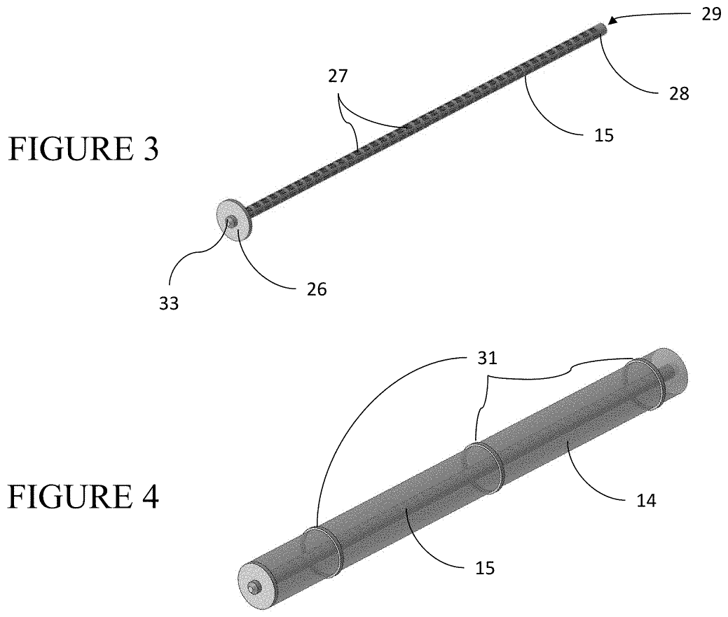

FIG. 3 and FIG. 4 show the components of the cell culture elements 12 in more detail. Specifically, FIG. 3 shows the support element 15, including perforations 27 and top flange 26. In FIG. 4, the cell culture substrate 14 is shown surrounding the support element 15. Optionally, bands 31 or other attachment mechanisms can be used to hold the cell culture substrate 14 tightly or securely to the support element 15. On top of the top flange 26, an alignment rod 33 can be provided, which can be inserted into the perforated outlet plate 25 to securing the cell culture elements 12.

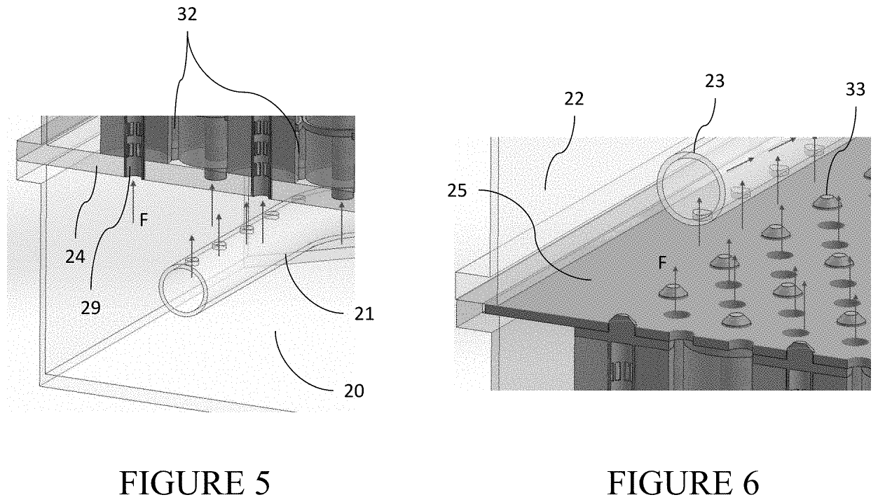

FIG. 5 is a detailed view of fluid flow from an inlet manifold 21 and/or inlet plenum 20 to the cell culture elements 12. Specifically, the support elements 15 are inserted into the perforated inlet plate 24 to fluidly connect the interior space 29 with the inlet plenum 20 and, thus, the inlet 16. Each perforation in the perforated inlet plate 24 is fluidly connected to an interior space 29 of a cell culture element to efficiently direct fluid flow (indicated by arrows) into the interior of the cell culture elements 12. Once in the interior space 29 and within the cell culture space 13, the fluid can then flow through the perforations 27 of the support element and spread through the cell culture substrate 14. Upon flowing through the cell culture substrate 14, the fluid enters an interstitial space 32 between cell culture elements 12. As shown in FIG. 6, the perforations in the perforated outlet plate 25 are open to this interstitial space 32 to allow media to exit the cell culture space 13 and flow to the outlet plenum 22 or outlet 17. While some of the perforations of the perforated outlet plate 25 are open to the interstitial space 32, other perforations are used to secure the alignment rod 33 of the support elements 15. According to embodiments, the top flange 26 seals the top of the interior space so that media does not flow out the top end of the support element, but rather is directed out radially through the perforations 27 in the outer wall 28 of the support elements 15.

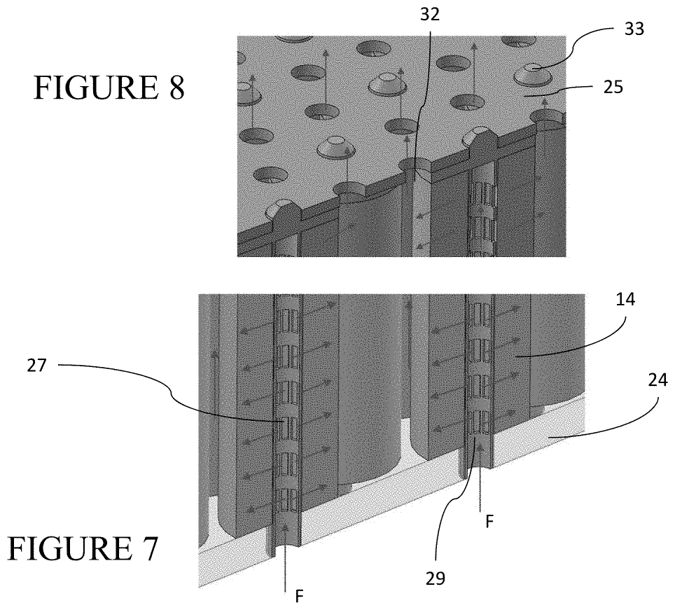

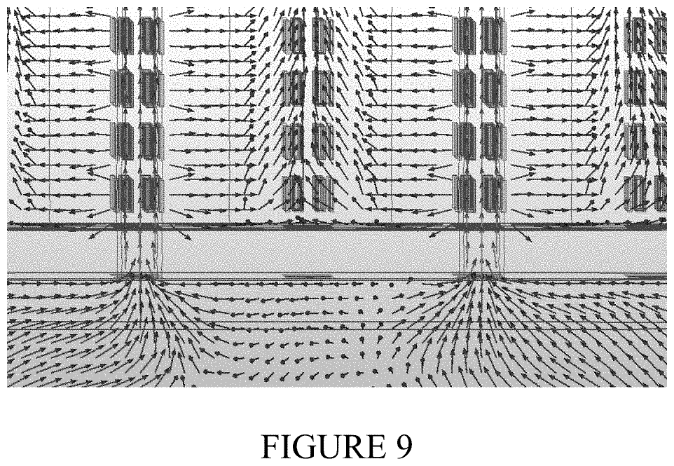

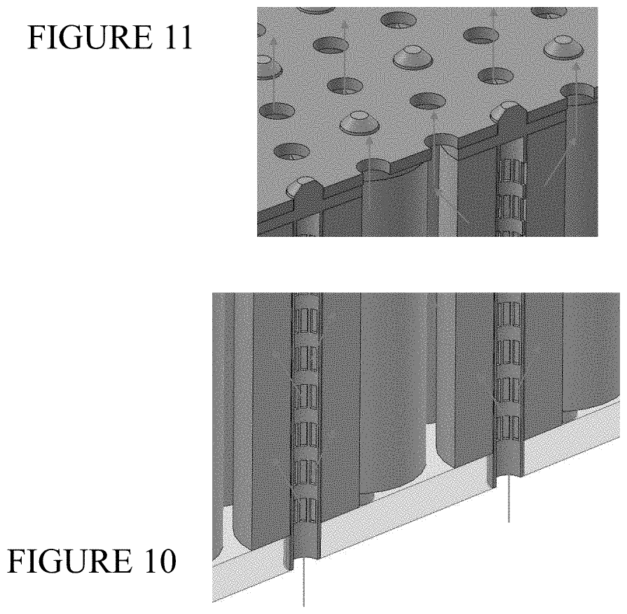

FIG. 7 and FIG. 8 show alternative views of the fluid flow (indicated by the arrows) as the fluid flows radially outward from the support elements 15 and through the cell culture substrate 14. In FIG. 9, this flow pattern is simulated, and it is shown that fluid flow through the area containing the cell culture substrate 14 is very uniform until it exists the cell culture substrate 14 and flows upward toward the outlet 17. FIG. 10 and FIG. 11 show yet another view of fluid flow through the cell culture substrate 14 where, instead of the fluid flowing horizontally through the cell culture substrate, it is angled upward. This directing of the fluid can be achieved by the design of the perforations 27.

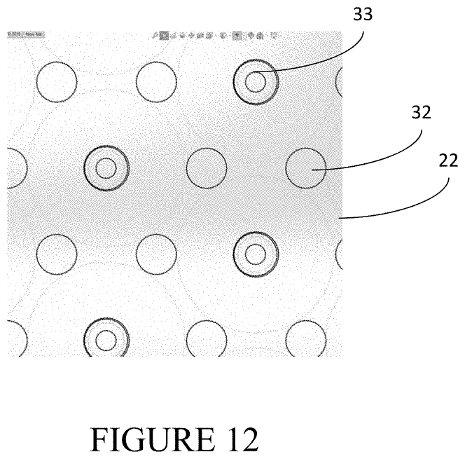

As discussed above, the perforated outlet plate 25 includes a plurality of perforations, some of which the alignment rods 13 are inserted into, and some of which serve as paths for fluid flow from the interstitial space 32 to the outlet plenum 22. An example of the arrangement of perforations is shown in FIG. 12.

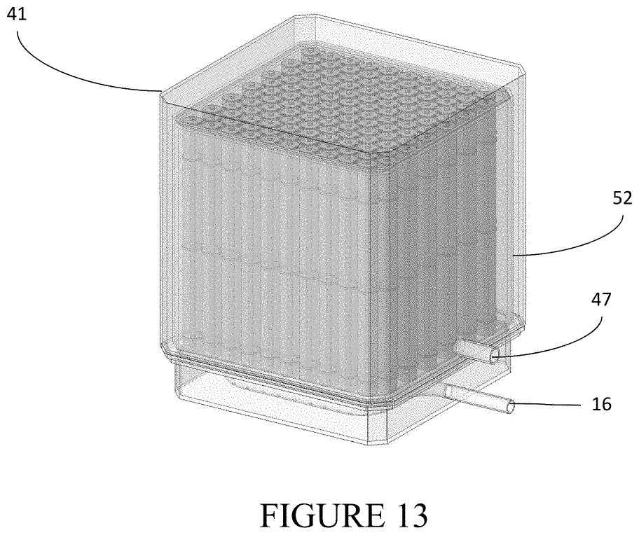

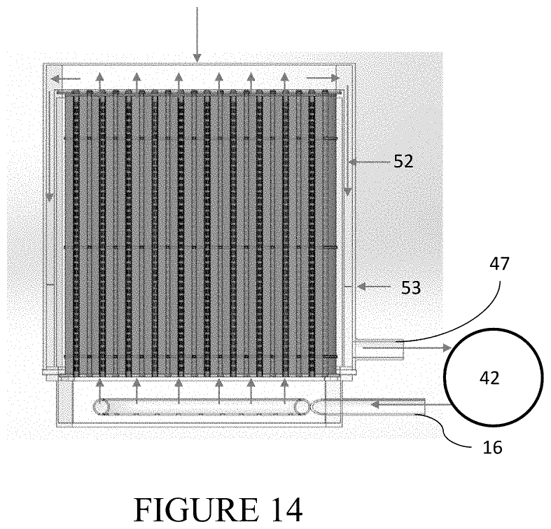

A modified embodiment of the cell culture system 10 discussed above is shown in FIG. 13. Specifically, similar to cell culture system 10, the cell culture system 40 includes bioreactor vessel 41 and at least one cell growth element 12. What differs in FIG. 13 is that the bioreactor vessel 41 has an outlet 47 disposed near the bottom of the bioreactor vessel 41, so positioned to drain media that exiting the top of the cell culture space and flowed over the side of the cell culture space into a spill-over space 52 at least partially surrounding the cell culture space where the media fills the spill-over space 52 to a fill level 53. A cross-section view of the cell culture system 40 is shown in FIG. 14. Also shown in FIG. 14 is a pump 42 fluidly connected to the inlet 16 and outlet 47. Thus, the pump 42 can recirculate fluid through the cell culture space. Although not shown in the above figures, it is contemplated that such a pump can be used in multiple embodiments of this disclosure, including those described above.

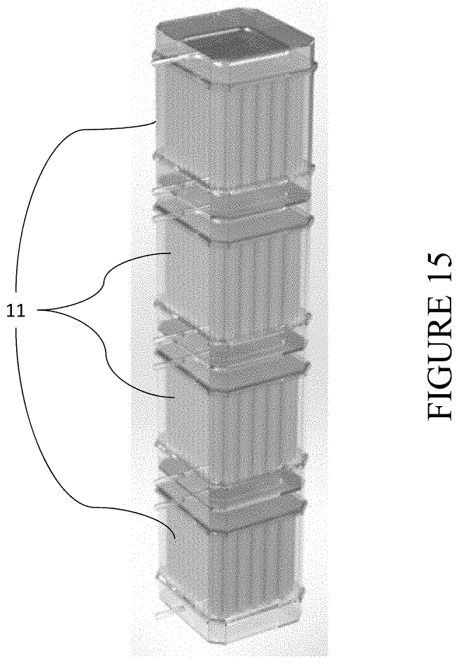

As discussed above, the modular design of the cell culture elements allows for the cell culture systems to be scaled to meet various requirements. Another advantage of the cell culture systems, according to some embodiments, is that multiple bioreactor vessels 11 can be stacked to provide even larger cell culture yields in a relatively small footprint, as shown in FIG. 15. In the stack of FIG. 15, each inlet can be driven by a separate pump, or they can be manifolded together. Similarly, the outlets can be manifolded together, or they can each flow to separate media conditioning vessels to allow for media conditioning on a per vessel level.

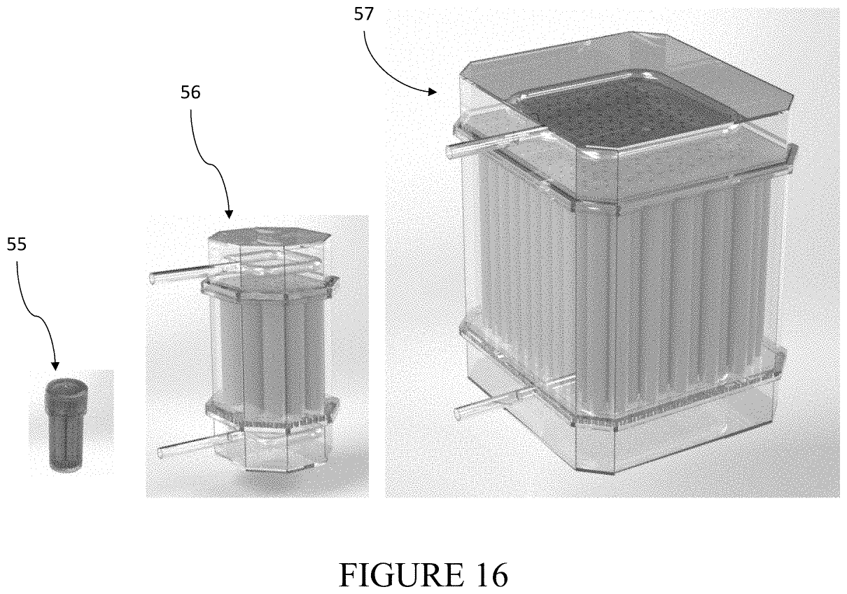

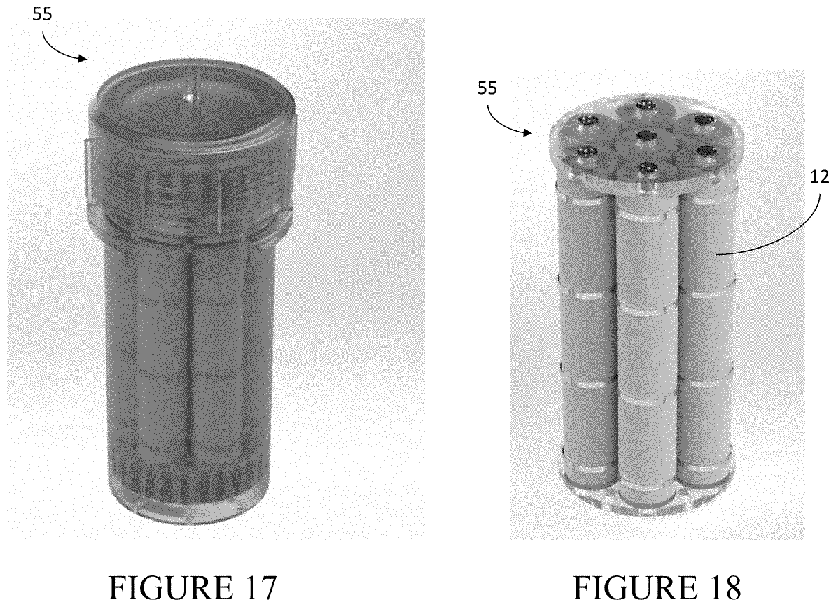

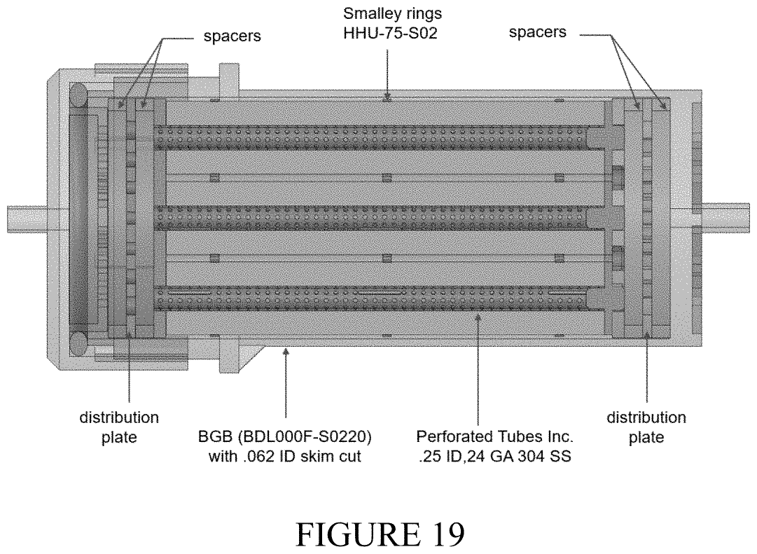

The size of vessels, and the corresponding number of cell culture elements, is also scalable, as shown in FIG. 16. It is contemplated that bioreactor vessel 55 can have an operating volume of, for example, 200 mL; bioreactor vessel 56 can have a volume of 3 L; and bioreactor vessel 57 can have a volume of 50 L. The advantage to scaling the bioreactor and not solely to number of cell culture elements 12 is that reducing the volume of the vessel, for example, also reduces the amount of media needed to perfuse it. However, because each individual cell culture element is constructed similarly and has a uniform structure, the performance of the cell culture system is predictable and scalable. The smaller bioreactor vessel 55 is shown in more detail in FIGS. 17, 18, and 19. The vessel 55 is sized to hold a maximum of seven cell culture elements 12, but otherwise operates similarly to the embodiments discussed above.

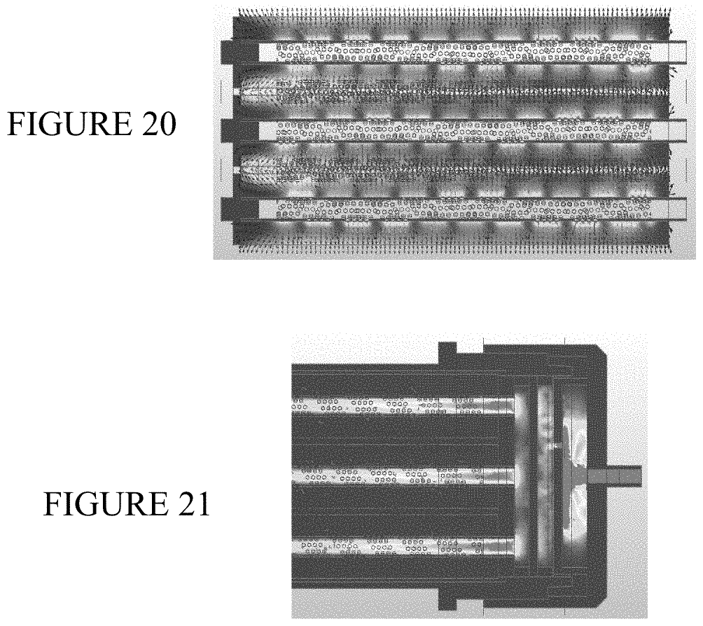

Modeling of the flow behavior for the bioreactor vessel 55 was performed and the results are shown in FIGS. 20 and 21. In FIG. 20, the flow vectors show predominately radial flow out of the cell culture elements. There is some elevated velocity at the perforations of the support elements, but this dissipates quickly, which is advantages to maintain healthy adhered cells. In FIG. 21, a detailed view of the inlet zone is shown. Distribution of media to each cell culture element is substantially uniform, which helps even distribution of cells, nutrients, and media.

One unique advantage of the embodiments disclosed herein is the use of the plurality of cell culture elements. The plurality of cell culture elements should provide for more uniform cell growth and flow fields compared to designs which use one, or a small number of larger bulk stack of substrate material. The cell growth elements are also easy to construct and deploy in the vessel. The geometry of the tubular elements can be optimized for performance, and can enable scaling to large or small size in a straightforward manner.

Cell Culture Substrate

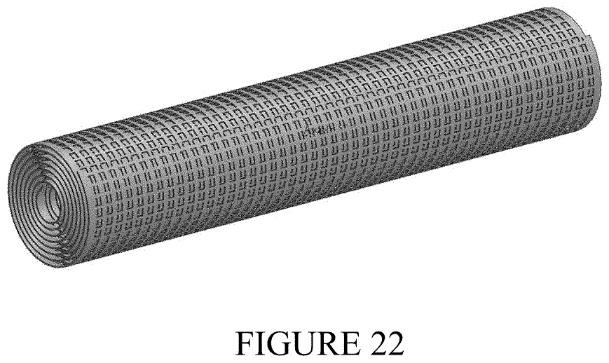

FIG. 22 shows an example of a roll of cell culture substrate material. As discussed herein, the substrate material can be a woven polymer material, in some preferred embodiments. However, embodiments are not limited to this construction. For example, the material can 3D-print, injection molded, stamped, fused from smaller pieces of filaments, or produced according to other methods known in the art. According to some of these methods, the substrate can be formed as a flat sheet and then rolled. Alternatively, the substrate can be formed into a three-dimensional cylinder, such as by 3D printing.

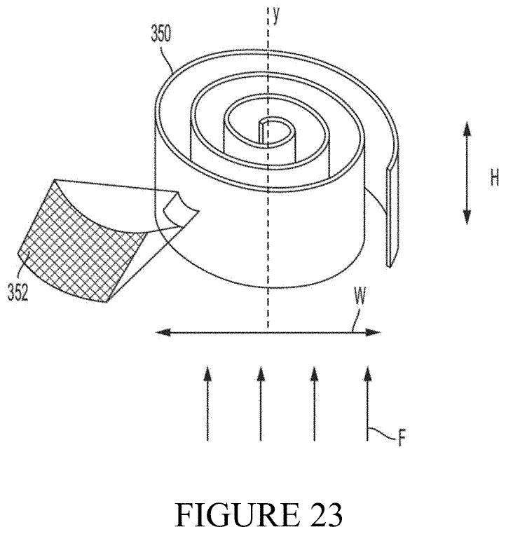

FIG. 23 shows an embodiment of the substrate in which the substrate is formed into a cylindrical roll 350. For example, a sheet of a substrate material that includes a mesh substrate 352 is rolled into a cylinder about a central longitudinal axis y. The cylindrical roll 350 has a width W along a dimension perpendicular to the central longitudinal axis y and a height H along a direction perpendicular to the central longitudinal axis y. In one or more preferred embodiments, the cylindrical roll 350 is designed to be within a bioreactor vessel such that the central longitudinal axis y is parallel to a direction of bulk flow F of fluid through the bioreactor or culture chamber that houses the cylindrical roll. In some embodiments, the support element may include one or more attachment sites for holding one or more portions of the cell culture substrate 352 at the inner part of the cylindrical roll. These attachment sites may be hooks, clasps, posts, clamps, or other means of attaching the mesh sheet to the support element. Alternatively, the substrate can be constricted by bands or another fastener surrounding the roll, or it can be attached to one or both of the perforated inlet plate or perforated outlet plate.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the substrate is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the substrate has a high surface-to-volume ratio for culturing anchorage dependent cells. According to various embodiments, the substrate can be arranged or packed in a bioreactor in certain ways discussed here for uniform cell seeding and growth, uniform media perfusion, and efficient cell harvest.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes per batch, or about 10¹⁶ to about 10¹⁹ viral genomes per batch, or about 10¹⁶ to about 10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

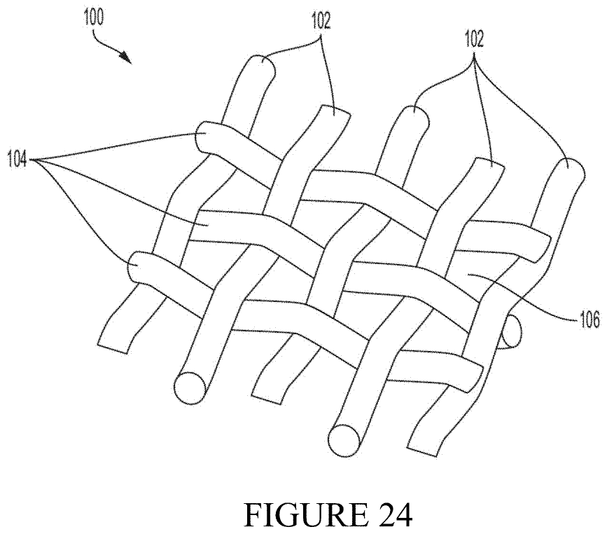

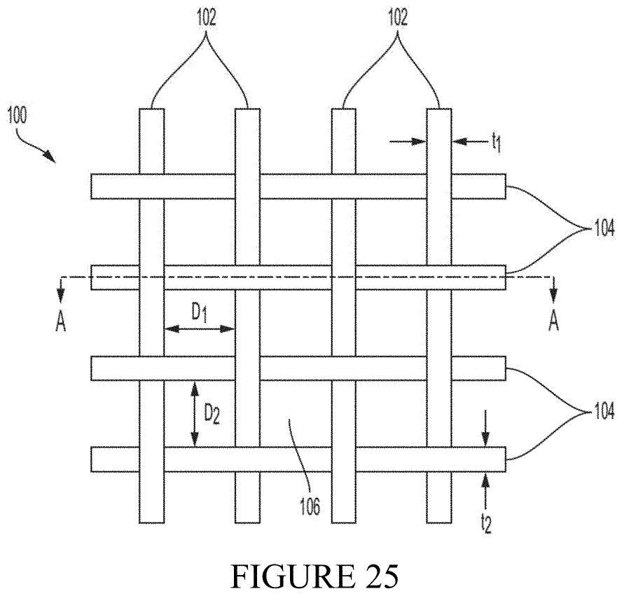

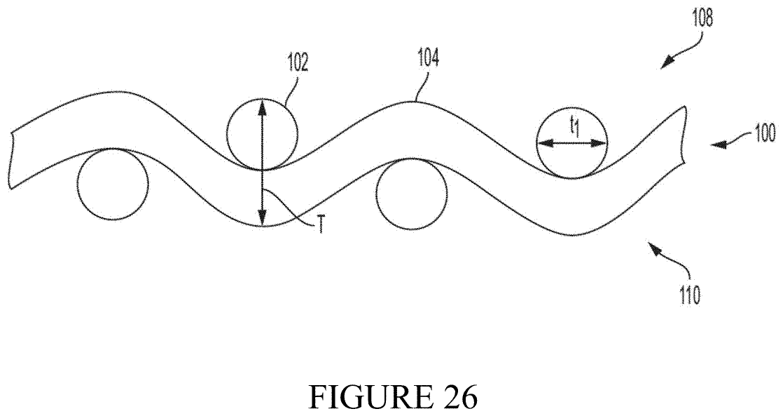

FIGS. 24 and 25 show a three-dimensional (3D) perspective view and a two-dimensional (2D) plan view, respectively, of a cell culture substrate 100, according to an example cell culture substrate of one or more embodiments of this disclosure. The cell culture substrate 100 is a woven mesh layer made of a first plurality of fibers 102 running in a first direction and a second plurality of fibers 104 running in a second direction. The woven fibers of the substrate 100 form a plurality of openings 106, which can be defined by one or more widths or diameters (e.g., D₁, D₂). The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Thus, as shown in FIG. 26, a thickness T of the woven mesh 100 may be thicker than the thickness of a single fiber (e.g., t₁). As used herein, the thickness T is the maximum thickness between a first side 108 and a second side 110 of the woven mesh. Without wishing to be bound by theory, it is believed that the three-dimensional structure of the substrate 100 is advantageous as it provides a large surface area for culturing adherent cells, and the structural rigidity of the mesh can provide a consistent and predictable cell culture substrate structure that enables uniform fluid flow.

In FIG. 25, the openings 106 have a diameter D₁, defined as a distance between opposite fibers 102, and a diameter D₂, defined as a distance between opposite fibers 104. D₁ and D₂ can be equal or unequal, depending on the weave geometry. Where D₁ and D₂ are unequal, the larger can be referred to as the major diameter, and the smaller as the minor diameter. In some embodiments, the diameter of an opening may refer to the widest part of the opening. Unless otherwise specified, the opening diameter, as used herein, will refer to a distance between parallel fibers on opposite sides of an opening.

A given fiber of the plurality of fibers 102 has a thickness t₁, and a given fiber of the plurality of fibers 104 has a thickness t₂. In the case of fibers of round cross-section, as shown in FIG. 24, or other three-dimensional cross-sections, the thicknesses t₁ and t₂ are the maximum diameters or thicknesses of the fiber cross-section. According to some embodiments, the plurality of fibers 102 all have the same thickness t₁, and the plurality of fiber 104 all have the same thickness t₂. In addition, t₁ and t₂ may be equal. However, in one or more embodiments, t₁ and t₂ are not equal such as when the plurality of fibers 102 are different from the plurality of fiber 104. In addition, each of the plurality of fibers 102 and plurality of fibers 104 may contain fibers of two or more different thicknesses (e.g., t₁ₐ, t_{1b}, etc., and t₂ₐ, t_{2b}, etc.). According to embodiments, the thicknesses t₁ and t₂ are large relative to the size of the cells cultured thereon, so that the fibers provide an approximation of a flat surface from the perspective of the cell, which can enable better cell attachment and growth as compared to some other solutions in which the fiber size is small (e.g., on the scale of the cell diameter). Due to three-dimensional nature of woven mesh, as shown in FIGS. 24-26, the 2D surface area of the fibers available for cell attachment and proliferation exceeds the surface area for attachment on an equivalent planar 2D surface.

In one or more embodiments, a fiber may have a diameter in a range of about 50 μm to about 1000 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm×100 μm to about 1000 μm×1000 μm. In some embodiments, the opening may have a diameter o about 50 μm to about 1000 μm; about 100 μm to about 750 μm; about 125 μm to about 600 μm; about 150 μm to about 500 μm; about 200 μm to about 400 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture substrate comprises a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture substrate includes a stack, roll, or other arrangement of overlapping substrate, the packing density of the cell culture substrate will impact the surface area of the packed bed substrate. Packing density can vary with the packing thickness of the substrate material (e.g., the space needed for a layer of the substrate). For example, if a stack of cell culture substrate has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture substrate with a lower density packing of layers (e.g., when higher permeability is a priority) or a higher density of packing (e.g., when maximizing substrate surface area is a priority). According to one or more embodiments, the packing thickness can be from about 50 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture substrate, whether of a single layer of cell culture substrate or of a cell culture substrate having multiple layers of substrate). For example, in a particular embodiment, a single layer of woven mesh substrate having a circular shape and diameter of 6 cm can have an effective surface area of about 68 cm². The “effective surface area,” as used herein, is the total surface area of fibers in a portion of substrate material that is available for cell attachment and growth. Unless stated otherwise, references to “surface area” refer to this effective surface area. According to one or more embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm². These ranges of effective surface area are provided for example only, and some embodiments may have different effective surface areas. The cell culture substrate can also be characterized in terms of porosity.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

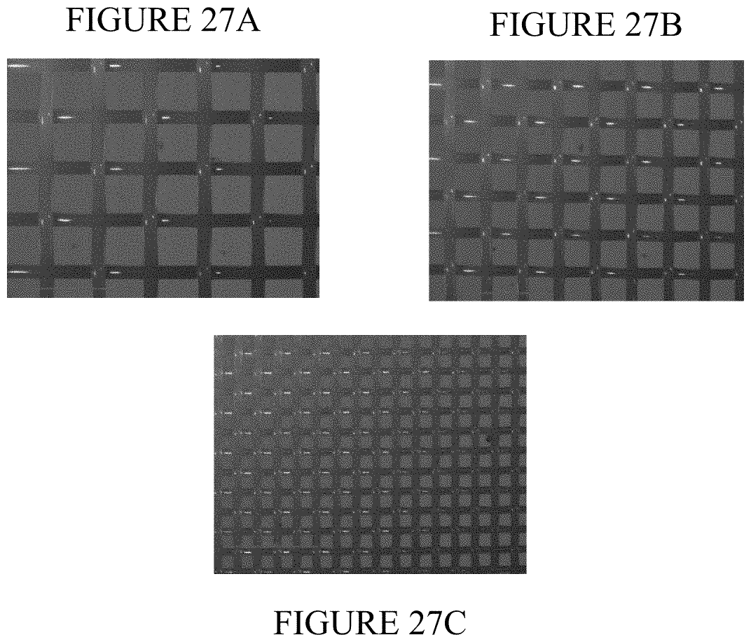

FIGS. 27A, 27B, and 27C show different examples of woven mesh according to some contemplated embodiments of this disclosure. The fiber diameter and opening size of these meshes are summarized in Table 1 below, as well as the approximate magnitude of increase in cell culture surface area provided by a single layer of the respective meshes relative to a comparable 2D surface. In Table 1, Mesh A refers to the mesh of FIG. 27A, Mesh B to the mesh of FIG. 27B, and Mesh C to the mesh of FIG. 27C. The three mesh geometries of Table 1 are examples only, and embodiments of this disclosure are not limited to these specific examples. Because Mesh C offers the highest surface area, it may be advantageous in achieving a high density in cell adhesion and proliferation, and thus provide the most efficient substrate for cell culturing. However, in some embodiments, it may be advantageous for the cell culture substrate to include a mesh with lower surface area, such as Mesh A or Mesh B, or a combination of meshes of different surface areas, to achieve a desired cell distribution or flow characteristics within the culture chamber, for example.

TABLE 1
Comparison of meshes in FIGS. 27A-27C, and the resulting
increase in cell culture surface area as compared to a 2D surface.

| | Mesh A | Mesh B | Mesh C |
|---|---|---|---|
| Fiber diameter | 273 ± 3 μm | 218 ± 3 μm | 158 ± 3 μm |
| Mesh opening | 790 × 790 μm | 523 × 523 μm | 244 × 244 μm |
| Surface area | ×1.6 | ×1.8 | ×2.5 |
| increase of one | | | |
| layer of mesh | | | |
| compared to | | | |
| 2D surface | | | |

As shown by the above table, the three-dimensional quality of the meshes provides increased surface area for cell attachment and proliferation compared to a planar 2D surface of comparable size. This increased surface area aids in the scalable performance achieved by embodiments of this disclosure. For process development and process validation studies, small-scale bioreactors are often required to save on reagent cost and increase experimental throughput. Embodiments of this disclosure are applicable to such small-scale studies, but can be scaled-up to industrial or production scale, as well. For example, if 100 layers of Mesh C in the form of 2.2 cm diameter circles are packed into a cylindrical packed bed with a 2.2 cm internal diameter, the total surface area available for cells to attach and proliferate is equal to about 935 cm². To scale such bioreactor ten times, one could use a similar setup of a cylindrical packed bed with 7 cm internal diameter and 100 layers of the same mesh. In such a case, the total surface area would be equal 9,350 cm². In some embodiments, the available surface area is about 99,000 cm²/L or more. Because of the plug-type perfusion flow in a packed bed, the same flow rate expressed in ml/min/cm² of cross-sectioned packed bed surface area can be used in smaller-scale and larger-scale versions of the bioreactor. Likewise, in the cell culture systems disclosed herein, the length and number of cell culture elements can be varied to adjust the available surface area. Also, it is contemplated that the amount of substrate on a given cell culture element (e.g., the thickness of the roll of cell culture substrate) can be varied for the same purpose. A larger surface area allows for higher seeding density and higher cell growth density. According to one or more embodiments, the cell culture substrate described herein has demonstrated cell seeding densities of up to 22,000 cells/cm² or more. For reference, the Corning HyperFlask® has a seeding density on the order of 20,000 cells/cm² on a two-dimensional surface.

Another advantage of the higher surface areas and high cell seeding or growing densities is that the cost of the embodiments disclosed herein can be the same or less than competing solution. Specifically, the cost per cellular product (e.g., per cell or per viral genome) can be equal to or less than other packed bed bioreactors.

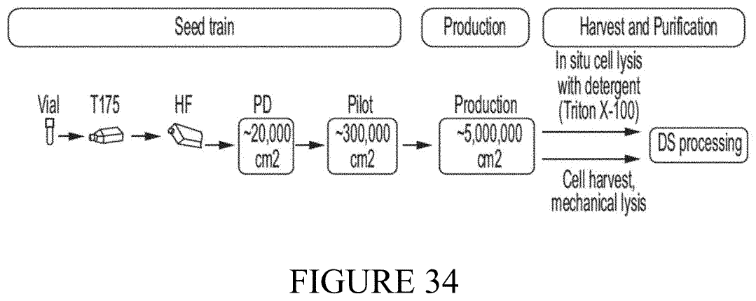

In a further embodiment of the present disclosure discussed below, a woven mesh substrate can be packed in a cylindrical roll format within the bioreactor. In such an embodiment, the scalability of the packed bed bioreactor can be achieved by increasing the overall length of the (unrolled) mesh strip and/or its width (e.g., the height of the roll). The amount of mesh used in this cylindrical roll configuration can vary based on the desired packing density of the packed bed. For example, the cylindrical rolls can be densely packed in a tight roll or loosely packed in a loose roll. The density of packing will often be determined by the required cell culture substrate surface area required for a given application or scale. In one embodiment, the required length of the mesh can be calculated from the packed bed bioreactor diameter by using following formula:

$$L = \frac{π ( {R}^{2} - {r}^{2} )}{t} \qquad Equation 1$$

where L is the total length of mesh required to pack the bioreactor (i.e., H in FIG. 34), R is the internal radius of packed bed culture chamber, r is the radius of an inner support around which mesh is rolled, and t is the thickness of one layer of the mesh. In such a configuration, scalability of the bioreactor can be achieved by increasing diameter or width (i.e., W in FIG. 34) of the packed bed cylindrical roll and/or increasing the height H of the packed bed cylindrical roll, thus providing more substrate surface area for seeding and growing adherent cells.

By using a structurally defined culture substrate of sufficient rigidity, high-flow-resistance uniformity across the substrate or packed bed is achieved. According to various embodiments, the substrate can be deployed in monolayer or multilayer formats. This flexibility eliminates diffusional limitations and provides uniform delivery of nutrients and oxygen to cells attached to the substrate. In addition, the open substrate lacks any cell entrapment regions in the packed bed configuration, allowing for complete cell harvest with high viability at the end of culturing. The substrate also delivers packaging uniformity for the packed bed and enables direct scalability from process development units to large-scale industrial bioprocessing unit. The ability to directly harvest cells from the packed bed eliminates the need of resuspending a substrate in a stirred or mechanically shaken vessel, which would add complexity and can inflict harmful shear stresses on the cells. Further, the high packing density of the cell culture substrate yields high bioprocess productivity in volumes manageable at the industrial scale.

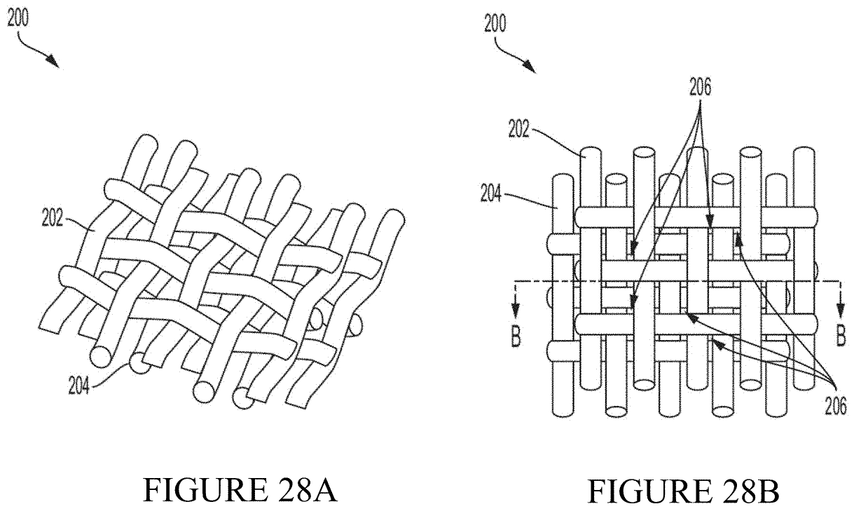

FIG. 28A shows an embodiment of the cell culture substrate comprising multiple layers of substrate 200, and FIG. 28B is a plan view of the same multilayer substrate 200. The multilayer substrate 200 includes a first mesh substrate layer 202 and a second mesh substrate layer 204. Despite the overlapping of the first and second substrate layers 202 and 204, the mesh geometries (e.g., ratio of opening diameters to fiber diameters) is such that the openings of the first and second substrate layers 202 and 204 overlap and provide paths for fluid to flow through the total thickness of the multilayer substrate 200, as shown by the filament-free openings 206 in FIG. 28B. These overlapping layers of substrate can comprise separate pieces of substrate material or single piece of material that has ben folded over or rolled around itself, as is the case in the cell culture elements described herein.

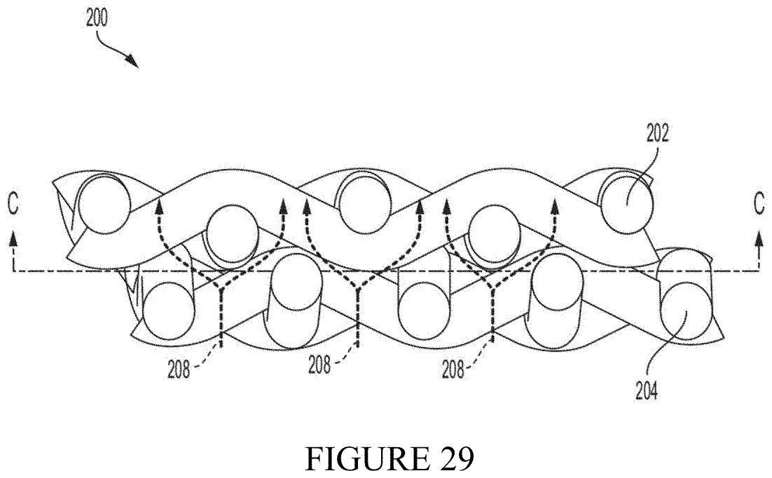

FIG. 40 shows a cross-section view of the multilayer substrate 200 at line B-B in FIG. 28B. The arrows 208 show the possible fluid flow paths through openings in the second substrate layer 204 and then around filaments in the first substrate layer 202. The geometry of the mesh substrate layers is designed to allow efficient and uniform flow through one or multiple substrate layers. In addition, the structure of the substrate 200 can accommodate fluid flow through the substrate in multiple orientations. For example, as shown in FIG. 29, the direction of bulk fluid flow (as shown by arrows 208) is perpendicular to the major side surfaces of the first and second substrate layers 202 and 204. However, the substrate can also be oriented with respect to the flow such that the sides of the substrate layers are parallel to the bulk flow direction.

According to embodiments of this disclosure, cell culture substrates are provided that exhibit, due at least in part to their uniform and open structure, essentially isotropic flow of media, cells, nutrients, etc. through the substrate. In contrast, substrates for adherent cells in existing bioreactors do not exhibit this behavior and instead their packed beds tend to create preferential flow channels and have substrate materials with anisotropic permeability. The flexibility of the substrate of the current disclosure allows for its use in various applications and bioreactor or container designs while enabling better and more uniform permeability throughout the bioreactor vessel.

According to some embodiments, the typical non-woven substrates used in commercially available cell culture systems have a much lower permeability of about 7.5×10⁻¹² m², which can be about 1/50 of the permeability across the open and/or woven substrates according to embodiments of this disclosure. For example, when the non-woven substrate material is cut into smaller strips and packed randomly, the permeability can increase enormously and became similar as open woven mesh. However, this increased permeability is believed to be the result of the flow mostly bypassing around the mesh strips due to the channeling effect discussed above. In other words, increasing the permeability of other packed beds can come at the cost of uniformity.

In the case of open woven mesh, the open structure allowed liquid to flow easily through the mesh and did not create a dead zone behind the open mesh layer. It is believed that the regular structure of the woven mesh also contributed to the uniform flow distribution through each layer of substrate material. This, in turn, enables more uniform flow in through the entire packed bed.

Permeability and residence time experiments have shown that the type of non-woven, irregular cell culture substrate used in current bioreactors has lower permeability than the substrates according to embodiments of the present disclosure. These non-woven or irregular substrates also have different permeability or flow rates depending on the direction of flow relative to the non-woven substrate, whereas the substrates of the present disclosure exhibits essentially isotropic flow behavior. Due to the non-uniform flow and lower residence time of the non-woven substrates, nutrients and transfection reagents can take longer to reach to the cells on the substrate surface or the other side of a substrate layer, as compared to the open, uniform substrates in some embodiments of the present disclosure. Adding to this is the higher permeability of the randomly packed non-woven substrate, which suggest a strong channeling effect and thus non-uniform delivery of cells or nutrients.

Culture System

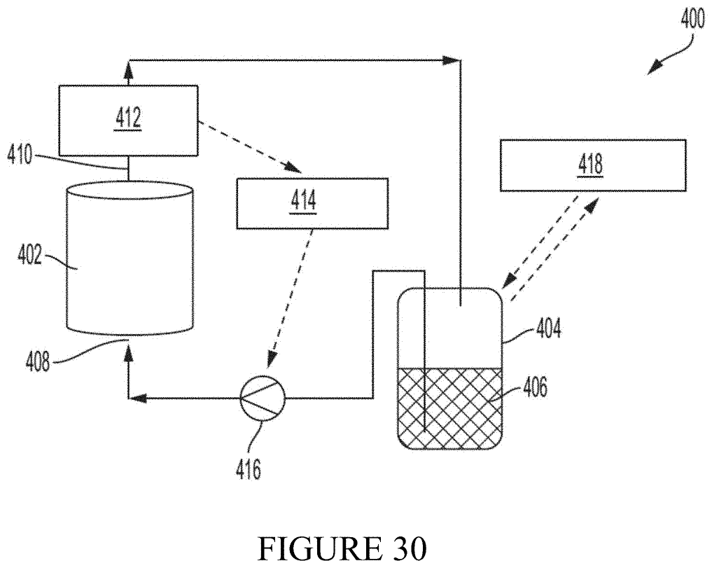

FIG. 30 shows a cell culture system 400 according to one or more embodiments. The system 400 includes a bioreactor 402 housing the cell culture elements according to one or more embodiments disclosed herein. The bioreactor 402 can be fluidly connected to a media conditioning vessel 404, and the system is capable of supplying a cell culture media 406 within the conditioning vessel 404 to the bioreactor 402. The media conditioning vessel 404 can include sensors and control components including, but not limited to, dissolved oxygen (DO) sensors, pH sensors, oxygenator/gas sparging unit, temperature probes, and nutrient addition and base addition ports. A gas mixture supplied to sparging unit can be controlled by a gas flow controller for N₂, O₂, and CO₂ gasses. The media conditioning vessel 404 also contains an impeller (not shown) for media mixing. All media parameters measured by sensors listed above can be controlled by a media conditioning control unit 418 in communication with the media conditioning vessel 404, and capable of measuring and/or adjusting the conditions of the cell culture media 406 to the desired levels. As shown in FIG. 30, the media conditioning vessel 404 is provided as a vessel that is separate from the bioreactor vessel 402. This can have advantages in terms of being able to condition the media separate from where the cells are cultured, and then supplying the conditioned media to the cell culture space. However, in some embodiments, media conditioning can be performed within the bioreactor vessel 402, such as in an inlet plenum or other compartment within the vessel.

The media from the media 406 conditioning vessel 404 is delivered to the bioreactor 402 via an inlet 408, which may also include an injection port for cell inoculum to seed and begin culturing of cells. The bioreactor vessel 402 may also include on or more outlets 410 through which the cell culture media 406 exits the vessel 402. In addition, cells or cell products may be output through the outlet 410 and/or the inlet 408. To analyze the contents of the outflow from the bioreactor 402, one or more sensors 412 may be provided in the line. In some embodiments, the system 400 includes a flow control unit 414 for controlling the flow into the bioreactor 402. For example, the flow control unit 414 may receive a signal from the one or more sensors 412 (e.g., an O₂ sensor) and, based on the signal, adjust the flow into the bioreactor 402 by sending a signal to a pump 416 (e.g., peristaltic pump) upstream of the inlet 408 to the bioreactor 402. Thus, based on one or a combination of factors measured by the sensors 412, the pump 416 can control the flow into the bioreactor 402 to obtain the desired cell culturing conditions.

The media perfusion rate is controlled by the signal processing unit 414 that collects and compares sensors signals from media conditioning vessel 404 and sensors located at the packed bed bioreactor outlet 410. Because of the pack flow nature of media perfusion through the packed bed bioreactor 402, nutrients, pH, and oxygen gradients are developed along the packed bed. The perfusion flow rate of the bioreactor can be automatically controlled by the flow control unit 414 operably connected to the peristaltic pump 416, according to the flow chart in FIG. 44.

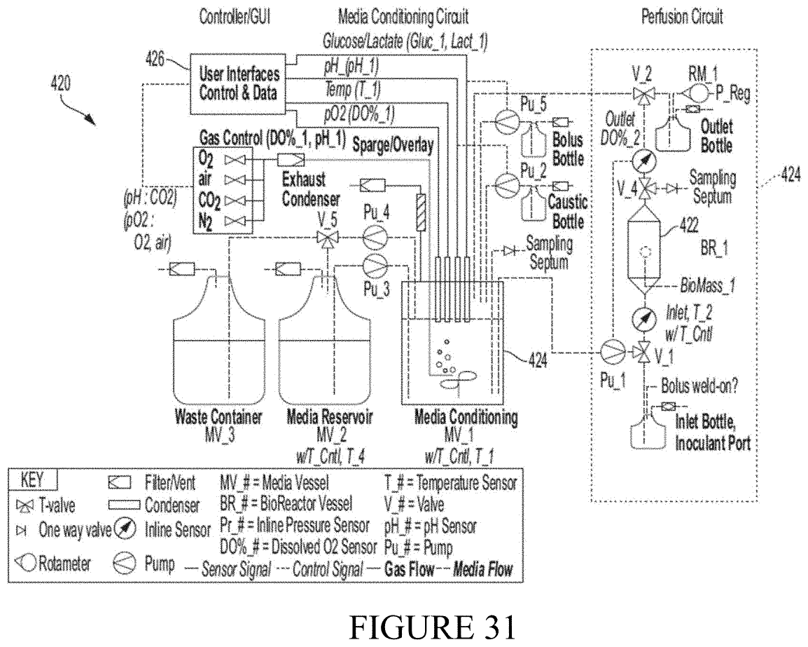

FIG. 31 shows a more detailed schematic of a cell culture system 420 according to one or more embodiments. The basic construction of the system 420 is similar to the system 400 in FIG. 30, with a packed bed bioreactor 422 having a vessel containing one or more cell culture elements with cell culture substrate material, such as a PET woven mesh, and a separate media conditioning vessel 424. In contrast to system 400, however, system 420 shows the details of the system, including sensors, user interface and controls, and various inlet and outlets for media and cells. According to some embodiments, the media conditioning vessel 424 is controlled by the controller 426 to provide the proper temperature, pH, O₂, and nutrients. While in some embodiments, the bioreactor 422 can also be controlled by the controller 426, in other embodiments the bioreactor 422 is provided in a separate perfusion circuit 428, where a pump is used to control the flow rate of media through the perfusion circuit 428 based on the detection of O₂ at or near the outlet of the bioreactor 422.

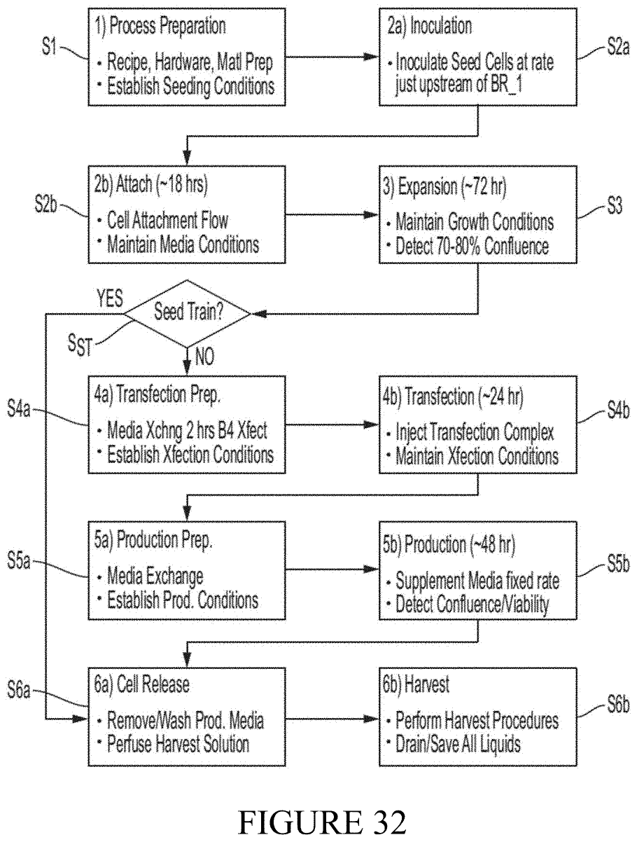

The systems of FIGS. 30 and 31 can be operated according to process steps according to one or more embodiments. As shown in FIG. 32, these process steps can include process preparation (S1), seeding and attaching cells (S2a, S2b), cell expansion (S3), transfection (S4a, S4b), production of viral vector (S5a, S5b), and cell release and harvesting (S6a, S6b).

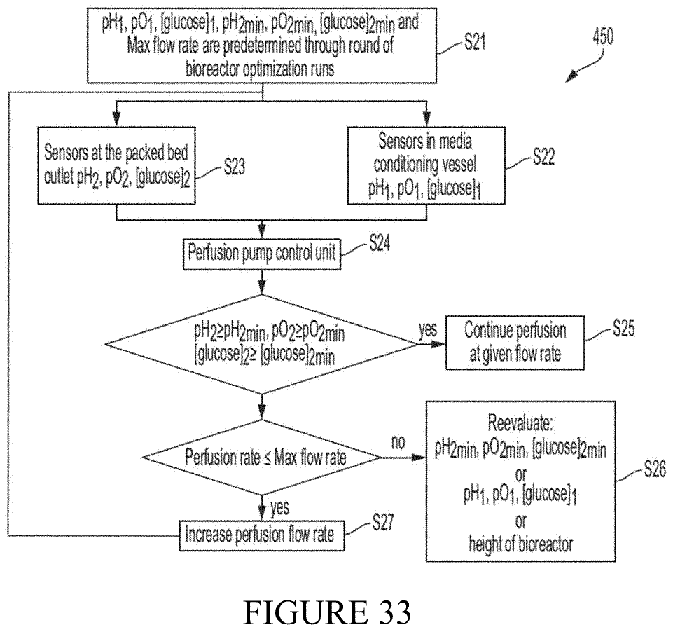

FIG. 33 shows an example of a method 450 for controlling the flow of a perfusion bioreactor system, such as the system 400 of FIG. 30 or 31. According to the method 450, certain parameters of the system 400 are predetermined at step S21 through bioreactor optimization runs. From these optimization runs, the values of pH₁, pO₁, [glucose]₁, pH₂, pO₂, [glucose]₂, and maximum flow rate can be determined. The values for pH₁, pO₁, and [glucose]₁ are measured within the cell culture chamber of the bioreactor 402 at step S22, and pH₂, pO₂, and [glucose]₂ are measured by sensors 412 at the outlet of the bioreactor vessel 402 at step S23. Based on these values at S22 and S23, a perfusion pump control unit makes determinations at S24 to maintain or adjust the perfusion flow rate. For example, a perfusion flow rate of the cell culture media to the cell culture chamber may be continued at a present rate if at least one of pH₂≥pH₂ₘᵢₙ, pO₂≥pO₂ₘᵢₙ, and [glucose]₂≥[glucose]₂ₘᵢₙ (S25). If the current flow rate is less than or equal to a predetermined max flow rate of the cell culture system, the perfusion flow rate is increased (S27). Further, if the current flow rate is not less than or equal to the predetermined max flow rate of the cell culture system, a controller of the cell culture system can reevaluate at least one of: (1) pH₂ₘᵢₙ, pO₂ₘᵢₙ, and [glucose]₂ₘᵢₙ; (2) pH₁, pO₁, and [glucose]₁; and (3) a height of the bioreactor vessel (S26).

The “cell culture chamber” or “defined culture space,” as used herein, refers to a space within the culture chamber occupied by the cell culture elements and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture substrate during the culturing of cells, and/or during the inflow or outflow of culture media to the culture chamber.

The bioreactor vessel optionally includes one or more outlets capable of being attached to inlet and/or outlet means. Through the one or more outlets, liquid, media, or cells can be supplied to or removed from the chamber. A single port in the vessel may act as both the inlet and outlet, or multiple ports may be provided for dedicated inlets and outlets.

The packed bed cell culture substrate of one or more embodiments can consist of the woven cell culture mesh substrate without any other form of cell culture substrate disposed in or interspersed with the cell culture substrate. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc. According to some embodiments, the cell culture elements include rolled or stacked layers of cell culture substrate creating a layered cell culture substrate, and no other solid material (e.g., spacer and/or other cell culture material) is disposed between adjacent layers).

As discussed herein, the cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

The embodiments disclosed herein have advantages over the existing platforms for cell culture and viral vector production. It is noted that the embodiments of this disclosure can be used for the production of a number of types of cells and cell byproducts, including, for example, adherent or semi-adherent cells, Human embryonic kidney (HEK) cells (such as HEK23), including transfected cells, viral vectors, such as Lentivirus (stem cells, CAR-T) and Adeno-associated virus (AAV). These are examples of some common applications for a bioreactor or cell culture substrate as disclosed herein, but are not intended to be limiting on the use or applications of the disclosed embodiments, as a person of ordinary skill in the art would understand the applicability of the embodiments to other uses.

As discussed above, one advantage of embodiments of this disclosure is the flow uniformity through the cell culture substrate. Without wishing to be bound by theory, it is believed that the regular or uniform structure of the cell culture substrate provides a consistent and uniform body through which media can flow. In contrast, existing platform predominately rely on irregular or random substrates, such as felt-like or non-woven fibrous materials.

### EXAMPLES

### Example 1

Table 2 shows the example substrates of some embodiments, where the substrates are made of woven PET mesh of various constructions.

TABLE 2
Example mesh substrates.

| | | Opening | Fiber | | Packing | Surface Area | Normalized |
|---|---|---|---|---|---|---|---|
| Mesh | Weave | Diameter | Diameter | Open | Thickness | of 60 mm | Surface to |
| Sample | Pattern | (μm) | (μm) | area | (μm) | disc (cm²) | Volume ratio |
| A | Plain | 250 | 160 | 37% | 280 | 74.9 | 1.00 |
| B | Twill | 250 | 152 | 39% | 280 | 74.2 | 0.99 |
| C | Plain | 210 | 147 | 35% | 230 | 80.9 | 1.16 |
| D | Plain | 200 | 112 | 41% | 130 | 68.1 | 1.30 |
| E | Plain | 300 | 195 | 37% | 370 | 68.1 | 0.83 |
| F | Plain | 319 | 128 | 51%* | 200 | 53.0 | 0.99 |

### Example 2

As discussed herein, the embodiments of this disclosure provide cell culture substrates, bioreactor systems, and methods of culturing cells or cell by-products that are scalable and can be used to provide a cell seed train to gradually increase a cell population. One problem in existing cell culture solutions is the inability for a given bioreactor system technology to be part of a seed train. Instead, cell populations are usually scaled up on various cell culture substrates. This can negatively impact the cell population, as it is believed that cells become acclimated to certain surfaces and being transferred to a different type of surface can lead to inefficiencies. Thus, it would be desirable to minimize such transitions between cell culture substrates or technologies. By using the same cell culture substrate across the seed train, as enabled by embodiments of this disclosure, efficiency of scaling up a cell population is increased. FIG. 34 shows an example of one or more embodiments where the woven cell culture substrate of the present application is used as part of a seed train to allow for a smaller bioreactor to seed a larger bioreactor. Specifically, as shown in FIG. 34, the seed train can begin with a vial of starter cells which are seeded into a first vessel (such as a T175 flask from Corning), then into a second vessel (such as a HyperFlask® from Corning), then into a process-development scale bioreactor system according to embodiments of this invention (effective surface area of substrate of about 20,000 cm²), and then into a larger bioreactor pilot system according to embodiments of this invention (effective surface area of substrate is about 300,000 cm²). At the end of this seed train, the cells can be seeded into a production-scale bioreactor vessel according to embodiments of this disclosure, with a surface area o about 5,000,000 cm², for example. Harvest and purification steps can then be performed when the cell culture is complete. As shown in FIG. 34, harvest can be accomplished via in situ cell lysis with a detergent (such as Triton X-100), or via mechanical lysis; and further downstream processing can be performed, as needed.

The benefits of using the same cell culture substrate within the seed train (e.g., from process development level to pilot level, or even to production level) include efficiencies gained from the cells being accustomed to the same surface during the seed train and production stages; a reduced number of manual, open manipulations during seed train phases; more efficient use of the packed bed due to uniform cell distribution and fluid flow, as described herein; and the flexibility of using mechanical or chemical lysis during viral vector harvest.

Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a cell culture system comprising a bioreactor vessel comprising an interior void defining a cell culture space, an inlet fluidly connected to a first end of the cell culture space, and an outlet fluidly connected to a second end of the cell culture space; and at least one cell growth element disposed in the cell culture space, the cell growth element comprising a cell culture substrate surrounding a support element extending in a direction from the first end to the second end of the cell culture space.

Aspect 2 pertains to the cell culture system of Aspect 1, wherein the cell culture substrate comprises a sheet of cell culture substrate material that is wrapped or wound around the support element.

Aspect 3 pertains to the cell culture system of Aspect 1 or Aspect 2, wherein the cell culture substrate comprises a woven substrate material comprising a plurality of interwoven fibers with surfaces configured for adhering cells thereto.

Aspect 4 pertains to the cell culture system of any of Aspects 1-3, further comprising a plurality of cell growth elements disposed in the cell culture space and aligned in the direction from the first end to the second end of the cell culture space.

Aspect 5 pertains to the cell culture system of Aspect 4, wherein the plurality of cell growth elements are removably attached to the cell culture space such that the cell culture system can accommodate various numbers of cell growth elements during cell culture.

Aspect 6 pertains to the cell culture system of any of Aspects 1-5, wherein the central support is tubular with a peripheral wall surrounding a hollow core, the peripheral wall comprising a plurality of perforations fluidly connecting an interior of the central support to an exterior of the central support.

Aspect 7 pertains to the cell culture system of Aspect 6, wherein the hollow core of the central support is fluidly connected to the inlet, and the cell culture system comprises a fluid flow path that comprises flowing from the inlet, then through the hollow core, then radially out from the central support through the plurality of perforations, then through the cell culture substrate, and then out through the outlet.

Aspect 8 pertains to the cell culture system of any of Aspects 1-7, further comprising an inlet plenum fluidly connected to and disposed between the inlet and the cell culture space.

Aspect 9 pertains to the cell culture system of Aspect 8, further comprising a perforated inlet plate disposed between the inlet plenum and the cell culture space, the perforated inlet plate comprising a plurality of perforations fluidly connecting the inlet plenum directly to the hollow core at a first end of the central support.

Aspect 10 pertains to the cell culture system of any of Aspects 1-9, further comprising an outlet plenum fluidly connected to and disposed between the cell culture space and the outlet.

Aspect 11 pertains to the cell culture system of Aspect 10, further comprising a perforated outlet plate disposed between the cell culture space and the outlet plenum, the perforated outlet plate comprising a plurality of perforations fluidly connecting a portion of the cell culture space comprising the exterior of the central support to the outlet plenum.

Aspect 12 pertains to the cell culture system of Aspect 11, wherein the central support is attached to a second end of the central support.

Aspect 13 pertains to the cell culture system of Aspect 12, wherein the hollow core is not open at the second end of the central support such that the hollow core is not directly fluidly connected to the outlet plenum via the second end of the central support.

Aspect 14 pertains to the cell culture system of any of Aspects 8-13, further comprising an inlet manifold disposed in the inlet plenum, the inlet manifold fluidly connected to the inlet and configured to distribute fluid evenly throughout the inlet plenum or evenly to the perforated inlet plate.

Aspect 15 pertains to the cell culture system of any of Aspects 10-14, further comprising an outlet manifold disposed in the outlet plenum, the outlet plenum fluidly connected to the outlet and configured to direct fluid exiting the cell culture space to the outlet.

Aspect 16 pertains to the cell culture system of any of Aspects 1-15, wherein the at least one cell culture element has a cylindrical shape.

Aspect 17 pertains to the cell culture system of any of Aspects 1-16, wherein the at least one cell culture element comprises an attachment means for attaching the cell culture substrate to the central support.

Aspect 18 pertains to the cell culture system of any of Aspects 1-17, wherein the cell culture space has a volume of at least about 50 mL, at least about 100 mL, at least about 200 mL, at least about 300 mL, at least about 500 mL, at least about 1 L, at least about 2 L, at least about 3 L, at least about 10 L, at least about 20 L, at least about 30 L, at least about 40 L, at least about 50 L, from about 50 mL to about 500 mL, from about 1 L to about 10 L, or from about 10 L to about 50 L.

Aspect 19 pertains to the cell culture system of any of Aspects 1-18, comprising from about 7 cell culture elements to about 130 cell culture elements.

Aspect 20 pertains to the cell culture system of any of Aspects 1-19, wherein the cell culture substrate comprises a stack or roll of cell culture substrate material without any other solid material between adjacent layers of the cell culture substrate.

Aspect 21 pertains to a cell culture system comprising: a bioreactor vessel comprising an interior void defining a cell culture space, an inlet fluidly connected to a first end of the cell culture space, and an outlet fluidly connected to a second end of the cell culture space; an inlet plenum fluidly connected to and disposed between the inlet and the cell culture space; an outlet plenum fluidly connected to and disposed between the cell culture space and the outlet; and a perforated inlet plate disposed between the inlet plenum and the cell culture space, the perforated inlet plate comprising at least one perforation, wherein the cell culture space is configured to house at least one cell growth element therein, the at least one cell growth element comprising a porous cell culture substrate surrounding a perforated central tube, and wherein the at least one perforation of the perforated inlet plate fluidly connects the inlet plenum directly to a hollow center of the perforated central tube when the at least one cell growth element is disposed in the cell culture space.

Aspect 22 pertains to the cell culture system of Aspect 21, further comprising a perforated outlet plate disposed between the cell culture space and the outlet plenum, the perforated outlet plate comprising at least one perforation, wherein the at least one perforation of the perforated outlet plate fluidly connects a portion of the cell culture space comprising an exterior of the perforated central tube when the at least one cell growth element is disposed in the cell culture space.

Aspect 23 pertains to the cell culture system of Aspect 22, wherein the perforated outlet plate comprising at least one attachment site for attaching the at least on cell culture element.

Aspect 24 pertains to the cell culture system of any of Aspects 21-23, further comprising an inlet manifold disposed in the inlet plenum, the inlet manifold fluidly connected to the inlet and configured to distribute fluid evenly throughout the inlet plenum or evenly to the perforated inlet plate.

Aspect 25 pertains to the cell culture system of any of Aspects 22-24, further comprising an outlet manifold disposed in the outlet plenum, the outlet plenum fluidly connected to the outlet and configured to direct fluid exiting the cell culture space to the outlet.

Aspect 26 pertains to the cell culture system of any of Aspects 21-25, wherein the cell culture vessel is configured to operate in culturing cells while housing any of a variety of numbers of cell culture elements.

Aspect 27 pertains to the cell culture system of any of Aspects 21-26, wherein the cell culture space has a volume of at least about 50 mL, at least about 100 mL, at least about 200 mL, at least about 300 mL, at least about 500 mL, at least about 1 L, at least about 2 L, at least about 3 L, at least about 10 L, at least about 20 L, at least about 30 L, at least about 40 L, at least about 50 L, from about 50 mL to about 500 mL, from about 1 L to about 10 L, or from about 10 L to about 50 L.

Aspect 28 pertains to the cell culture system of any of Aspects 21-27, wherein the cell culture space is configured to house from about 7 cell culture elements to about 130 cell culture elements.

Aspect 29 pertains to a method of culturing cells or cell products using the cell culture system of any of Aspects 1-20.

Aspect 30 pertains to the method of Aspect 29, the method comprising: providing the cell culture system; seeding cells on the cell culture substrate; flowing cell culture media through the cell culture system to culture the cells; and harvesting a product of the culturing of the cells.

Aspect 31 pertains to the method of Aspect 30, wherein the flowing of cell culture media through the cell culture system comprises: flowing the cell culture media into the cell culture space via the inlet; flowing the cell culture media from the inlet to an interior of the support element; flowing the cell culture media outward radially from the interior of the support element and through the cell culture substrate to a portion of the cell culture space exterior to the cell culture element; and flowing the cell culture media from the portion of the cell culture space out through the outlet.

Aspect 32 pertains to the method of any of Aspects 29-31, wherein harvesting the product of the culturing of the cells comprises harvesting greater than about 10¹⁴ viral genomes per batch, greater than about 10¹⁵ viral genomes per batch, greater than about 10¹⁶ viral genomes per batch, greater than about 10¹⁷ viral genomes per batch, up to or greater than about 10¹⁶ viral genomes per batch, about 10¹⁵ to about 10¹⁸ viral genomes per batch, about 10¹⁵ to about 10¹⁶ viral genomes per batch, about 10¹⁶ to about 10¹⁹ viral genomes per batch, about 10¹⁶ to about 10¹⁸ viral genomes per batch, about 10¹⁷ to about 10¹⁹ viral genomes per batch, about 10¹⁸ to about 10¹⁹ viral genomes per batch, or about 10¹⁸ or more viral genomes per batch.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

What is claimed:

1. A cell culture system comprising:
a bioreactor vessel comprising an interior void defining a cell culture space, an inlet fluidly connected to a first end of the cell culture space, and an outlet fluidly connected to a second end of the cell culture space; and
at least one cell growth element disposed in the cell culture space, the cell growth element comprising a cell culture substrate surrounding a support element extending in a direction from the first end to the second end of the cell culture space.

2. The cell culture system of claim 1, wherein the cell culture substrate comprises a sheet of cell culture substrate material that is wrapped or wound around the support element.

3. The cell culture system of claim 1 or claim 2, wherein the cell culture substrate comprises a woven substrate material comprising a plurality of interwoven fibers with surfaces configured for adhering cells thereto.

4. The cell culture system of any of claims 1-3, further comprising a plurality of cell growth elements disposed in the cell culture space and aligned in the direction from the first end to the second end of the cell culture space.

5. The cell culture system of claim 4, wherein the plurality of cell growth elements are removably attached to the cell culture space such that the cell culture system can accommodate various numbers of cell growth elements during cell culture.

6. The cell culture system of any of claims 1-5, wherein the central support is tubular with a peripheral wall surrounding a hollow core, the peripheral wall comprising a plurality of perforations fluidly connecting an interior of the central support to an exterior of the central support.

7. The cell culture system of claim 6, wherein the hollow core of the central support is fluidly connected to the inlet, and the cell culture system comprises a fluid flow path that comprises flowing from the inlet, then through the hollow core, then radially out from the central support through the plurality of perforations, then through the cell culture substrate, and then out through the outlet.

8. The cell culture system of any of claims 1-7, further comprising an inlet plenum fluidly connected to and disposed between the inlet and the cell culture space.

9. The cell culture system of claim 8, further comprising a perforated inlet plate disposed between the inlet plenum and the cell culture space, the perforated inlet plate comprising a plurality of perforations fluidly connecting the inlet plenum directly to the hollow core at a first end of the central support.

10. The cell culture system of any of claims 1-9, further comprising an outlet plenum fluidly connected to and disposed between the cell culture space and the outlet.

11. The cell culture system of claim 10, further comprising a perforated outlet plate disposed between the cell culture space and the outlet plenum, the perforated outlet plate comprising a plurality of perforations fluidly connecting a portion of the cell culture space comprising the exterior of the central support to the outlet plenum.

12. The cell culture system of claim 11, wherein the central support is attached to a second end of the central support.

13. The cell culture system of claim 12, wherein the hollow core is not open at the second end of the central support such that the hollow core is not directly fluidly connected to the outlet plenum via the second end of the central support.

14. The cell culture system of any of claims 8-13, further comprising an inlet manifold disposed in the inlet plenum, the inlet manifold fluidly connected to the inlet and configured to distribute fluid evenly throughout the inlet plenum or evenly to the perforated inlet plate.

15. The cell culture system of any of claims 10-14, further comprising an outlet manifold disposed in the outlet plenum, the outlet plenum fluidly connected to the outlet and configured to direct fluid exiting the cell culture space to the outlet.

16. The cell culture system of any of claims 1-15, wherein the at least one cell culture element has a cylindrical shape.

17. The cell culture system of any of claims 1-16, wherein the at least one cell culture element comprises an attachment means for attaching the cell culture substrate to the central support.

18. The cell culture system of any of claims 1-17, wherein the cell culture space has a volume of at least about 50 mL, at least about 100 mL, at least about 200 mL, at least about 300 mL, at least about 500 mL, at least about 1 L, at least about 2 L, at least about 3 L, at least about 10 L, at least about 20 L, at least about 30 L, at least about 40 L, at least about 50 L, from about 50 mL to about 500 mL, from about 1 L to about 10 L, or from about 10 L to about 50 L.

19. The cell culture system of any of claims 1-18, comprising from about 7 cell culture elements to about 130 cell culture elements.

20. The cell culture system of any of claims 1-19, wherein the cell culture substrate comprises a stack or roll of cell culture substrate material without any other solid material between adjacent layers of the cell culture substrate.

21. A cell culture vessel comprising:
a bioreactor vessel comprising an interior void defining a cell culture space, an inlet fluidly connected to a first end of the cell culture space, and an outlet fluidly connected to a second end of the cell culture space;
an inlet plenum fluidly connected to and disposed between the inlet and the cell culture space;
an outlet plenum fluidly connected to and disposed between the cell culture space and the outlet; and
a perforated inlet plate disposed between the inlet plenum and the cell culture space, the perforated inlet plate comprising at least one perforation,
wherein the cell culture space is configured to house at least one cell growth element therein, the at least one cell growth element comprising a porous cell culture substrate surrounding a perforated central tube, and
wherein the at least one perforation of the perforated inlet plate fluidly connects the inlet plenum directly to a hollow center of the perforated central tube when the at least one cell growth element is disposed in the cell culture space.

22. The cell culture vessel of claim 21, further comprising a perforated outlet plate disposed between the cell culture space and the outlet plenum, the perforated outlet plate comprising at least one perforation,
wherein the at least one perforation of the perforated outlet plate fluidly connects a portion of the cell culture space comprising an exterior of the perforated central tube when the at least one cell growth element is disposed in the cell culture space.

23. The cell culture vessel of claim 22, wherein the perforated outlet plate comprising at least one attachment site for attaching the at least on cell culture element.

24. The cell culture vessel of any of claims 21-23, further comprising an inlet manifold disposed in the inlet plenum, the inlet manifold fluidly connected to the inlet and configured to distribute fluid evenly throughout the inlet plenum or evenly to the perforated inlet plate.

25. The cell culture vessel of any of claims 22-24, further comprising an outlet manifold disposed in the outlet plenum, the outlet plenum fluidly connected to the outlet and configured to direct fluid exiting the cell culture space to the outlet.

26. The cell culture vessel of any of claims 21-25, wherein the cell culture vessel is configured to operate in culturing cells while housing any of a variety of numbers of cell culture elements.

27. The cell culture vessel of any of claims 21-26, wherein the cell culture space has a volume of at least about 50 mL, at least about 100 mL, at least about 200 mL, at least about 300 mL, at least about 500 mL, at least about 1 L, at least about 2 L, at least about 3 L, at least about 10 L, at least about 20 L, at least about 30 L, at least about 40 L, at least about 50 L, from about 50 mL to about 500 mL, from about 1 L to about 10 L, or from about 10 L to about 50 L.

28. The cell culture vessel of any of claims 21-27, wherein the cell culture space is configured to house from about 7 cell culture elements to about 130 cell culture elements.

29. A method of culturing cells or cell products using the cell culture system of any of claims 1-20.

30. The method of claim 29, the method comprising:
providing the cell culture system;
seeding cells on the cell culture substrate;
flowing cell culture media through the cell culture system to culture the cells; and
harvesting a product of the culturing of the cells.
